(19) United States
(12) Patent Application Publication
(10) Pub. No.: US 2025/0272691 A1
(43) Pub. Date: Aug. 28, 2025

(54) SYSTEM AND METHOD FOR VALUE BASED ADJUDICATION AND BLOCKCHAIN, HYBRID PROOF OF ACHIEVEMENT, AND PROOF OF ACKNOWLEDGEMENT

(71) Applicant: Healthy Engage LLC, Frisco, TX (US)

(72) Inventor: Ravi Seshadri, Frisco, TX (US)

(21) Appl. No.: 18/905,226

(22) Filed: Oct. 3, 2024

Related U.S. Application Data
(60) Provisional application No. 63/558,760, filed on Feb. 28, 2024.

(51) Int. Cl. G06Q 20/40; G06Q 20/38

(52) U.S. Cl. CPC G06Q 20/4016; G06Q 20/38215; G06Q 20/3825

(57) ABSTRACT

A method includes retrieving a plurality of data from one or more data source. The method also includes performing, using an adjudication and reasoning engine, adjudication with respect to the plurality of data and using at least one machine learning model. The method also includes outputting adjudication results from the at least one machine learning model. The method also includes generating a validity block based on the adjudication results. The method also includes appending the validity block to a blockchain. The method also includes receiving results of one or more hybrid transactions using the plurality of data. The method also includes determining, using the adjudication and reasoning engine, a proof of achievement score based on the results of the one or more hybrid transactions. The method also includes performing analysis and adjudication using the transaction repository and the at least one smart contract and determining a proof of acknowledgement result.

### CROSS-REFERENCE TO RELATED APPLICATION AND PRIORITY CLAIM

This application claims priority under 35 U.S.C. § 119 (e) to U.S. Provisional Patent Application No. 63/558,760 filed on Feb. 28, 2024, which is hereby incorporated by reference in its entirety.

### TECHNICAL FIELD

This disclosure relates generally to blockchain and machine learning systems. More specifically, this disclosure relates to a system and method for value based adjudication and blockchain, hybrid proof of achievement, and proof of acknowledgement.

### BACKGROUND

The healthcare industry is rapidly evolving and so are the options available to support it. However, current technological systems for monitoring patients and aspects of their healthcare are antiquated and rely on incomplete data and poor decision-making solutions.

### SUMMARY

This disclosure relates to a system and method for value based adjudication and blockchain, hybrid proof of achievement, and proof of acknowledgement.

In one aspect, a system includes at least one processing device. The at least one processing device is configured to retrieve a plurality of data from one or more data sources. The at least one processing device is also configured to perform, using an adjudication and reasoning engine, adjudication with respect to the plurality of data and using at least one machine learning model. The at least one processing device is also configured to output adjudication results from the at least one machine learning model. The at least one processing device is also configured to generate a validity block based on the adjudication results. The at least one processing device is also configured to append the validity block to a blockchain. The at least one processing device is also configured to receive results of one or more hybrid transactions stored in a transaction repository using the plurality of data. The at least one processing device is also configured to determine, using the adjudication and reasoning engine, a proof of achievement score based on the results of the one or more hybrid transactions. The at least one processing device is also configured to perform analysis and adjudication using the transaction repository and the at least one smart contract. The at least one processing device is also configured to determine and output a proof of acknowledgement result based on the analysis and adjudication.

In another aspect, a method includes retrieving a plurality of data from one or more data sources. The method also includes performing, using an adjudication and reasoning engine, adjudication with respect to the plurality of data and using at least one machine learning model. The method also includes outputting adjudication results from the at least one machine learning model. The method also includes generating a validity block based on the adjudication results. The method also includes appending the validity block to a blockchain. The method also includes receiving results of one or more hybrid transactions stored in a transaction repository using the plurality of data. The method also includes determining, using the adjudication and reasoning engine, a proof of achievement score based on the results of the one or more hybrid transactions. The method also includes performing analysis and adjudication using the transaction repository and the at least one smart contract. The method also includes determining and outputting a proof of acknowledgement result based on the analysis and adjudication.

Other technical features may be readily apparent to one skilled in the art from the following figures, descriptions, and claims.

Before undertaking the DETAILED DESCRIPTION below, it may be advantageous to set forth definitions of certain words and phrases used throughout this patent document. The terms “transmit,” “receive,” and “communicate,” as well as derivatives thereof, encompass both direct and indirect communication. The terms “include” and “comprise,” as well as derivatives thereof, mean inclusion without limitation. The term “or” is inclusive, meaning and/or. The phrase “associated with,” as well as derivatives thereof, means to include, be included within, interconnect with, contain, be contained within, connect to or with, couple to or with, be communicable with, cooperate with, interleave, juxtapose, be proximate to, be bound to or with, have, have a property of, have a relationship to or with, or the like.

Moreover, various functions described below can be implemented or supported by one or more computer programs, each of which is formed from computer readable program code and embodied in a computer readable medium. The terms “application” and “program” refer to one or more computer programs, software components, sets of instructions, procedures, functions, objects, classes, instances, related data, or a portion thereof adapted for implementation in a suitable computer readable program code. The phrase “computer readable program code” includes any type of computer code, including source code, object code, and executable code. The phrase “computer readable medium” includes any type of medium capable of being accessed by a computer, such as read only memory (ROM), random access memory (RAM), a hard disk drive, a compact disc (CD), a digital video disc (DVD), or any other type of memory. A “non-transitory” computer readable medium excludes wired, wireless, optical, or other communication links that transport transitory electrical or other signals. A non-transitory computer readable medium includes media where data can be permanently stored and media where data can be stored and later overwritten, such as a rewritable optical disc or an erasable memory device.

As used here, terms and phrases such as “have,” “may have,” “include,” or “may include” a feature (like a number, function, operation, or component such as a part) indicate the existence of the feature and do not exclude the existence of other features. Also, as used here, the phrases “A or B,” “at least one of A and/or B,” or “one or more of A and/or B” may include all possible combinations of A and B. For example, “A or B,” “at least one of A and B,” and “at least one of A or B” may indicate all of (1) including at least one A, (2) including at least one B, or (3) including at least one A and at least one B. Further, as used here, the terms “first” and “second” may modify various components regardless of importance and do not limit the components. These terms are only used to distinguish one component from another. For example, a first user device and a second user device may indicate different user devices from each other, regardless of the order or importance of the devices. A first component may be denoted a second component and vice versa without departing from the scope of this disclosure.

It will be understood that, when an element (such as a first element) is referred to as being (operatively or communicatively) “coupled with/to” or “connected with/to” another element (such as a second element), it can be coupled or connected with/to the other element directly or via a third element. In contrast, it will be understood that, when an element (such as a first element) is referred to as being “directly coupled with/to” or “directly connected with/to” another element (such as a second element), no other element (such as a third element) intervenes between the element and the other element.

As used here, the phrase “configured (or set) to” may be interchangeably used with the phrases “suitable for,” “having the capacity to,” “designed to,” “adapted to,” “made to,” or “capable of” depending on the circumstances. The phrase “configured (or set) to” does not essentially mean “specifically designed in hardware to.” Rather, the phrase “configured to” may mean that a device can perform an operation together with another device or parts. For example, the phrase “processor configured (or set) to perform A, B, and C” may mean a generic-purpose processor (such as a CPU or application processor) that may perform the operations by executing one or more software programs stored in a memory device or a dedicated processor (such as an embedded processor) for performing the operations.

The terms and phrases as used here are provided merely to describe some embodiments of this disclosure but not to limit the scope of other embodiments of this disclosure. It is to be understood that the singular forms “a,” “an,” and “the” include plural references unless the context clearly dictates otherwise. All terms and phrases, including technical and scientific terms and phrases, used here have the same meanings as commonly understood by one of ordinary skill in the art to which the embodiments of this disclosure belong. It will be further understood that terms and phrases, such as those defined in commonly-used dictionaries, should be interpreted as having a meaning that is consistent with their meaning in the context of the relevant art and will not be interpreted in an idealized or overly formal sense unless expressly so defined here. In some cases, the terms and phrases defined here may be interpreted to exclude embodiments of this disclosure.

Examples of an “electronic device” according to embodiments of this disclosure may include at least one of a smartphone, a tablet personal computer (PC), a mobile phone, a video phone, an e-book reader, a desktop PC, a laptop computer, a netbook computer, a workstation, a personal digital assistant (PDA), a portable multimedia player (PMP), an MP3 player, a mobile medical device, a camera, or a wearable device (such as smart glasses, a head-mounted device (HMD), electronic clothes, an electronic bracelet, an electronic necklace, an electronic accessory, an electronic tattoo, a smart mirror, or a smart watch).

Other examples of an electronic device include a smart home appliance. Examples of the smart home appliance may include at least one of a television, a digital video disc (DVD) player, an audio player, a refrigerator, an air conditioner, a cleaner, an oven, a microwave oven, a washer, a dryer, an air cleaner, a set-top box, a home automation control panel, a security control panel, a TV box (such as APPLETV or GOOGLE TV), a smart speaker or speaker with an integrated digital assistant (such as APPLE HOMEPOD or AMAZON ECHO), a gaming console (such as an XBOX, PLAYSTATION, or NINTENDO consoles), an electronic dictionary, an electronic key, a camcorder, or an electronic picture frame. Still other examples of an electronic device include at least one of various medical devices (such as diverse portable medical measuring devices (like a blood sugar measuring device, a heartbeat measuring device, or a body temperature measuring device), a magnetic resource angiography (MRA) device, a magnetic resource imaging (MRI) device, a computed tomography (CT) device, an imaging device, or an ultrasonic device), a navigation device, a global positioning system (GPS) receiver, an event data recorder (EDR), a flight data recorder (FDR), an automotive infotainment device, a sailing electronic device (such as a sailing navigation device or a gyro compass), avionics, security devices, vehicular head units, industrial or home robots, automatic teller machines (ATMs), point of sales (POS) devices, or Internet of Things (IoT) devices (such as a bulb, various sensors, electric or gas meter, sprinkler, fire alarm, thermostat, street light, toaster, fitness equipment, hot water tank, heater, or boiler). Other examples of an electronic device include at least one part of a piece of furniture or building/structure, an electronic board, an electronic signature receiving device, a projector, or various measurement devices (such as devices for measuring water, electricity, gas, or electromagnetic waves). Note that, according to various embodiments of this disclosure, an electronic device may be one or a combination of the above-listed devices. The electronic device disclosed here is not limited to the above-listed devices and may include any other electronic devices now known or later developed.

In the following description, electronic devices are described with reference to the accompanying drawings, according to various embodiments of this disclosure. As used here, the term “user” may denote a human or another device (such as an artificial intelligent electronic device) using the electronic device.

Definitions for other certain words and phrases may be provided throughout this patent document. Those of ordinary skill in the art should understand that in many if not most instances, such definitions apply to prior as well as future uses of such defined words and phrases.

None of the description in this application should be read as implying that any particular element, step, or function is an essential element that must be included in the claim scope. The scope of patented subject matter is defined only by the claims. Moreover, none of the claims is intended to invoke 35 U.S.C. § 112 (f) unless the exact words “means for” are followed by a participle. Use of any other term, including without limitation “mechanism,” “module,” “device,” “unit,” “component,” “element,” “member,” “apparatus,” “machine,” “system,” “processor,” or “controller,” within a claim is understood by the Applicant to refer to structures known to those skilled in the relevant art and is not intended to invoke 35 U.S.C. § 112 (f).

### BRIEF DESCRIPTION OF THE DRAWINGS

For a more complete understanding of this disclosure and its advantages, reference is now made to the following description taken in conjunction with the accompanying drawings, in which like reference numerals represent like parts:

FIG. 1 illustrates an example network configuration including an electronic device in accordance with this disclosure;

FIG. 2 illustrates an example adjudication system architecture in accordance with this disclosure;

FIG. 3 illustrates an example method for value based adjudication in accordance with this disclosure;

FIG. 4 illustrates an example hybrid proof of achievement architecture in accordance with this disclosure;

FIG. 5 illustrates an example method for hybrid proof of achievement in accordance with this disclosure;

FIG. 6 illustrates an example validation and adjudication architecture in accordance with this disclosure;

FIG. 7 illustrates an example validation and adjudication method in accordance with this disclosure;

FIG. 8 illustrates an example proof of acknowledgement architecture in accordance with this disclosure; and

FIG. 9 illustrates an example proof of acknowledgement method in accordance with this disclosure.

### DETAILED DESCRIPTION

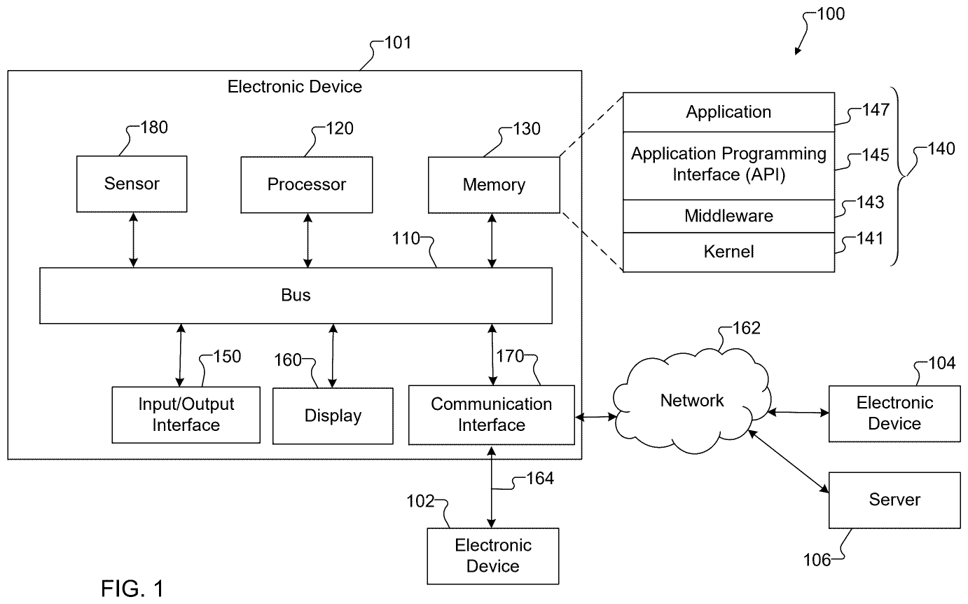

FIGS. 1 through 9, discussed below, and the various embodiments of this disclosure are described with reference to the accompanying drawings. However, it should be appreciated that this disclosure is not limited to these embodiments, and all changes and/or equivalents or replacements thereto also belong to the scope of this disclosure. The same or similar reference denotations may be used to refer to the same or similar elements throughout the specification and the drawings.

As noted above, the healthcare industry is rapidly evolving and so are the options available to support it. However, current technological systems for monitoring patients and aspects of their healthcare are antiquated and rely on incomplete data and poor decision-making solutions.

In various embodiments, this disclosure provides for system architectures and methods for validating transactions and performing adjudication using AI models and blockchain technology to ensure that all entities are held responsible for the overall outcome of patient health.

Cryptocurrencies have been developed in recent years, which operate on the principle of applying proof-of-work (POW) principles to process cryptocurrency transactions that are bound together in large blocks of data. The device that successfully meets the proof-of-work requirements (i.e., generating a double hash value with a required number of leading zero bits) for the transaction block and has their block accepted by peers receives a reward in the form of cryptocurrency.

An example of using proof-of-work outside the field of cryptocurrency includes using proof-of-work in prevent direct denial of service (DDOS) attacks, such as using a computer client to generate a POW to access a service where the POW could include hashing a message until a desired number of leading bit-level zeros is found, similar to the POW of some cryptocurrencies.

Another example of use of POW is in granting access to services, such as generating a cryptographic puzzle if no authentication token is included with a service request to run trialware. The client making the request must solve the cryptographic puzzle in order to receive authentication to proceed with running the trialware. However, previous systems did not provide for architectures and methods for validating transactions and performing healthcare adjudication using AI models and blockchain technology to ensure that all entities are held responsible for the overall outcome of patient health.

In various embodiments, this disclosure also provides an AI-powered distributed consensus system and method for hybrid (human and machine) transactions, focusing on rapidity, irreversibility, and agreement. The system and method provides for an enhanced POA approach by incorporating advanced AI techniques and enabling autonomous recommendation of agreement to foster a robust ecosystem of participants. The POA system and process involves all interested parties exchanging relevant activities, while a sophisticated reasoning engine facilitates the generation of a POA score, signifying relative achievement with respect to the original agreement or contract.

In various embodiments, this disclosure also provides a distributed consensus and monitoring method for evaluating healthcare needs by combining patient health and financial and engagement data addressing financial frictions in healthcare value-based care using a distributed consensus and monitoring method that utilizes patient health, financial, and engagement data. Utilizing advanced AI techniques, blockchain technology, and a proof of acknowledgement (PoA) mechanism, the system reduces financial friction, provides enhanced efficiency, and promotes transparency in the healthcare sector.

FIG. 1 illustrates an example network configuration 100 including an electronic device in accordance with this disclosure. The embodiment of the network configuration 100 shown in FIG. 1 is for illustration only. Other embodiments of the network configuration 100 could be used without departing from the scope of this disclosure.

According to embodiments of this disclosure, an electronic device 101 is included in the network configuration 100. The electronic device 101 can include at least one of a bus 110, a processor 120, a memory 130, an input/output (I/O) interface 150, a display 160, a communication interface 170, or a sensor 180. In some embodiments, the electronic device 101 may exclude at least one of these components or may add at least one other component. The bus 110 includes a circuit for connecting the components 120-180 with one another and for transferring communications (such as control messages and/or data) between the components.

The processor 120 includes one or more processing devices, such as one or more microprocessors, microcontrollers, digital signal processors (DSPs), application specific integrated circuits (ASICs), or field programmable gate arrays (FPGAs). In some embodiments, the processor 120 includes one or more of a central processing unit (CPU), an application processor (AP), a communication processor (CP), or a graphics processor unit (GPU). The processor 120 is able to perform control on at least one of the other components of the electronic device 101 and/or perform an operation or data processing relating to communication or other functions.

The memory 130 can include a volatile and/or non-volatile memory. For example, the memory 130 can store commands or data related to at least one other component of the electronic device 101. According to embodiments of this disclosure, the memory 130 can store software and/or a program 140. The program 140 includes, for example, a kernel 141, middleware 143, an application programming interface (API) 145, and/or an application program (or “application”) 147. At least a portion of the kernel 141, middleware 143, or API 145 may be denoted an operating system (OS).

The kernel 141 can control or manage system resources (such as the bus 110, processor 120, or memory 130) used to perform operations or functions implemented in other programs (such as the middleware 143, API 145, or application 147). The kernel 141 provides an interface that allows the middleware 143, the API 145, or the application 147 to access the individual components of the electronic device 101 to control or manage the system resources. These functions can be performed by a single application or by multiple applications that each carries out one or more of these functions. The middleware 143 can function as a relay to allow the API 145 or the application 147 to communicate data with the kernel 141, for instance. A plurality of applications 147 can be provided. The middleware 143 is able to control work requests received from the applications 147, such as by allocating the priority of using the system resources of the electronic device 101 (like the bus 110, the processor 120, or the memory 130) to at least one of the plurality of applications 147. The API 145 is an interface allowing the application 147 to control functions provided from the kernel 141 or the middleware 143.

The I/O interface 150 serves as an interface that can, for example, transfer commands or data input from a user or other external devices to other component(s) of the electronic device 101. The I/O interface 150 can also output commands or data received from other component(s) of the electronic device 101 to the user or the other external device.

The display 160 includes, for example, a liquid crystal display (LCD), a light emitting diode (LED) display, an organic light emitting diode (OLED) display, a quantum-dot light emitting diode (QLED) display, a microelectromechanical systems (MEMS) display, or an electronic paper display. The display 160 can also be a depth-aware display, such as a multi-focal display. The display 160 is able to display, for example, various contents (such as text, images, videos, icons, or symbols) to the user. The display 160 can include a touchscreen and may receive, for example, a touch, gesture, proximity, or hovering input using an electronic pen or a body portion of the user.

The communication interface 170, for example, is able to set up communication between the electronic device 101 and an external electronic device (such as a first electronic device 102, a second electronic device 104, or a server 106). For example, the communication interface 170 can be connected with a network 162 or 164 through wireless or wired communication to communicate with the external electronic device. The communication interface 170 can be a wired or wireless transceiver or any other component for transmitting and receiving signals, such as images.

The electronic device 101 further includes one or more sensors 180 that can meter a physical quantity or detect an activation state of the electronic device 101 and convert metered or detected information into an electrical signal. For example, one or more sensors 180 can include one or more cameras or other imaging sensors for capturing images of scenes. The sensor(s) 180 can also include one or more buttons for touch input, one or more microphones, a gesture sensor, a gyroscope or gyro sensor, an air pressure sensor, a magnetic sensor or magnetometer, an acceleration sensor or accelerometer, a grip sensor, a proximity sensor, a color sensor (such as an RGB sensor), a bio-physical sensor, a temperature sensor, a humidity sensor, an illumination sensor, an ultraviolet (UV) sensor, an electromyography (EMG) sensor, an electroencephalogram (EEG) sensor, an electrocardiogram (ECG) sensor, an infrared (IR) sensor, an ultrasound sensor, an iris sensor, or a fingerprint sensor. The sensor(s) 180 can further include an inertial measurement unit, which can include one or more accelerometers, gyroscopes, and other components. In addition, the sensor(s) 180 can include a control circuit for controlling at least one of the sensors included here. Any of these sensor(s) 180 can be located within the electronic device 101.

The first external electronic device 102 or the second external electronic device 104 can be a wearable device or an electronic device-mountable wearable device (such as an HMD). When the electronic device 101 is mounted in the electronic device 102 (such as the HMD), the electronic device 101 can communicate with the electronic device 102 through the communication interface 170. The electronic device 101 can be directly connected with the electronic device 102 to communicate with the electronic device 102 without involving with a separate network. The electronic device 101 can also be an augmented reality wearable device, such as eyeglasses, that include one or more cameras.

The wireless communication is able to use at least one of, for example, long term evolution (LTE), long term evolution-advanced (LTE-A), 5th generation wireless system (5G), millimeter-wave or 60 GHz wireless communication, Wireless USB, code division multiple access (CDMA), wideband code division multiple access (WCDMA), universal mobile telecommunication system (UMTS), wireless broadband (WiBro), or global system for mobile communication (GSM), as a cellular communication protocol. The wired connection can include, for example, at least one of a universal serial bus (USB), high definition multimedia interface (HDMI), recommended standard 232 (RS-232), or plain old telephone service (POTS). The network 162 includes at least one communication network, such as a computer network (like a local area network (LAN) or wide area network (WAN)), Internet, or a telephone network.

The first and second external electronic devices 102 and 104 and server 106 each can be a device of the same or a different type from the electronic device 101. According to certain embodiments of this disclosure, the server 106 includes a group of one or more servers. Also, according to certain embodiments of this disclosure, all or some of the operations executed on the electronic device 101 can be executed on another or multiple other electronic devices (such as the electronic devices 102 and 104 or server 106). Further, according to certain embodiments of this disclosure, when the electronic device 101 should perform some function or service automatically or at a request, the electronic device 101, instead of executing the function or service on its own or additionally, can request another device (such as electronic devices 102 and 104 or server 106) to perform at least some functions associated therewith.

The other electronic device (such as electronic devices 102 and 104 or server 106) is able to execute the requested functions or additional functions and transfer a result of the execution to the electronic device 101. The electronic device 101 can provide a requested function or service by processing the received result as it is or additionally. To that end, a cloud computing, distributed computing, or client-server computing technique may be used, for example. While FIG. 1 shows that the electronic device 101 includes the communication interface 170 to communicate with the external electronic device 104 or server 106 via the network 162, the electronic device 101 may be independently operated without a separate communication function according to some embodiments of this disclosure.

The server 106 can include the same or similar components as the electronic device 101 (or a suitable subset thereof). The server 106 can support to drive the electronic device 101 by performing at least one of operations (or functions) implemented on the electronic device 101. For example, the server 106 can include a processing module or processor that may support the processor 120 implemented in the electronic device 101.

Although FIG. 1 illustrates one example of a network configuration 100 including an electronic device 101, various changes may be made to FIG. 1. For example, the network configuration 100 could include any suitable number of each component in any suitable arrangement. In general, computing and communication systems come in a wide variety of configurations, and FIG. 1 does not limit the scope of this disclosure to any particular configuration. Also, while FIG. 1 illustrates one operational environment in which various features disclosed in this patent document can be used, these features could be used in any other suitable system.

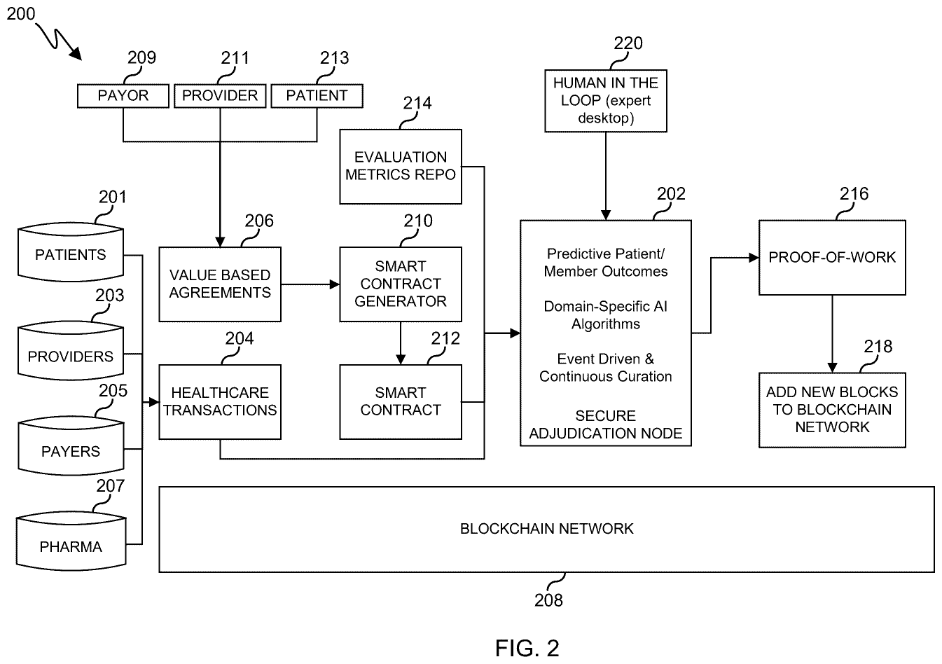

FIG. 2 illustrates an example adjudication system architecture 200 in accordance with this disclosure. For ease of explanation, the architecture 200 shown in FIG. 2 is described as being implemented on or supported by the electronic device 101 in the network configuration 100 of FIG. 1. However, the architecture 200 shown in FIG. 2 could be used with any other suitable device(s) and in any other suitable system(s), such as when the architecture 200 is implemented on or supported by the server 106.

As shown in FIG. 2, the architecture 200 provides for an AI-driven “value based” healthcare transaction adjudication systems that can carry out various associated methods. Most, if not all, healthcare transaction adjudications are only provided at an activity level, but the architecture 200 and associated methods shift the focus to the value delivered by healthcare services, thereby promoting more effective and efficient care, while implementing various AI elements to streamline the process and optimize decision-making. The architecture 200 also includes using blockchain technology, smart contracts, and AI to improve the evaluation and compensation of healthcare services.

As shown in FIG. 2, the architecture 200 includes a secure adjudication node or device 202 and a blockchain network 208. The secure adjudication node 202 can be provided data derived from various data sources involved in healthcare transactions 204, including patient sources 201, healthcare provider sources 203, healthcare payer sources 205, and healthcare pharmaceutical sources 207. The data sources may have their data stored in the form of databases. The secure adjudication node 202, in various embodiments, is a decentralized node within the blockchain network 208 that is responsible for evaluating healthcare transactions 204 based on the value delivered. The adjudication node maps the contractual nature of healthcare transactions to the agreed-upon value criteria and assigns ratings accordingly.

The architecture 200 is configured to receive information on healthcare transactions 204 associated with an item of mutual agreement via smart contracts. That is, the contracts can represent value based agreements 206 entered into between at least one payor 209, at least one provider 211, and at least one patient 213. These agreements 206 are then processed by a smart contract generator 210 to create one or more smart contracts 212. Value based healthcare transactions 204 associated with an item of mutual agreement via smart contracts are sent to an adjudication node 202. That is, data on these transactions, including the smart contracts 212 and data from an evaluation metrics repository 214, are provided to the adjudication node 202, which evaluates them based on the defined value criteria established by the parameters outlined in the smart contracts 212. In various embodiments, the item of mutual agreement constitutes a defined value delivered in the context of a care activity that uses a pharmaceutical drug. The set of healthcare transactions that define value could be a combined with a set of healthcare parameters (e.g., patient diagnosis, disease state, treatment protocols, Rx drugs, clinical evidence, outcomes, etc.).

The adjudication node 202 employs AI algorithms and machine learning techniques to analyze the transactions and assign ratings accordingly. These advanced AI algorithms and machine learning techniques are employed by the adjudication node 202 to analyze healthcare transactions, extract relevant data, and optimize the adjudication process to assist with identifying patterns and trends in healthcare parameters, predicting patient outcomes, and determining the efficacy of treatments. The adjudication node 202 maps the contractual nature of healthcare transactions associated with a stakeholder to the contractual value agreement. The adjudication node 202 awards criteria ratings for the transactions.

The adjudication node 202 assigns ratings to the transactions and validates their legitimacy. Upon establishing the validity of the transactions, the adjudication node 202 generates a new block using a proof-of-work principle 216. The proof-of-work principle 216 is a consensus mechanism used to validate healthcare transactions and create new blocks in the blockchain. The proof-of-work principle 216 uses nodes in the network to solve complex computational problems, preventing fraud and maintaining the integrity of the system. This new block is then appended, via a block appending operation 218 of the secure adjudication node 202, to the stakeholder's smart contract blockchain in the blockchain network 208, ensuring a secure and transparent record of value-based healthcare transactions. By incorporating additional AI elements, such as natural language processing, predictive analytics, reinforcement learning, data visualization, AI-driven consensus algorithms, and conversational AI, the adjudication system architecture 200 offers a comprehensive, intelligent, and efficient solution for evaluating and compensating healthcare services based on the value delivered, ultimately promoting better patient outcomes and more efficient resource allocation.

In the architecture 200, the smart contracts 212 are digitally enforced agreements between stakeholders (e.g., patients, healthcare providers, insurers, and pharmaceutical companies) that define the terms and conditions for value-based compensation. Smart contracts store essential parameters, such as patient diagnosis, disease state, treatment protocols, prescribed drugs, clinical evidence, and outcomes. In the architecture 200, the blockchain network 208 is a distributed, tamper-proof ledger that securely records healthcare transactions and smart contract data. The blockchain network ensures transparency, trust, and immutability in the adjudication process, facilitating collaboration among stakeholders.

The proof-of-work principle 216 is a healthcare-specific business level proof-of-work principle that is incorporated into the blockchain system/network 208 for value-based healthcare transaction adjudication. This proof-of-work mechanism focuses on evaluating the value delivered in healthcare services, leveraging domain-specific knowledge, AI-driven insights, and real-world healthcare parameters to validate transactions and maintain the integrity of the system. In various embodiments, the proof-of-work 216 for the healthcare-specific blockchain system performed by the adjudication node 202 includes domain-specific evaluation metrics (which can be included in the evaluation metrics repository), which can utilize a set of predefined, healthcare-specific evaluation metrics that reflect the value delivered by healthcare services. These metrics may include patient satisfaction, treatment success rates, cost-effectiveness, and adherence to best practice guidelines. In various embodiments, the proof-of-work 216 for the healthcare-specific blockchain system performed by the adjudication node 202 includes AI-driven insights, which include AI algorithms and machine learning techniques that analyze healthcare transactions and associated data to generate insights and determine the value of the transactions. These insights help the system to understand the impact of healthcare services on patient outcomes, ensuring that the proof-of-work mechanism remains focused on delivering value.

In various embodiments, the proof-of-work 216 for the healthcare-specific blockchain system performed by the adjudication node 202 includes real-world healthcare parameters, which can include incorporating in the proof-of-work mechanism real-world healthcare parameters (e.g., patient diagnosis, disease state, treatment protocols, prescribed drugs, clinical evidence, and outcomes) to evaluate the value of healthcare transactions, ensuring that the evaluation process is grounded in actual patient care and treatment experiences.

In various embodiments, the proof-of-work 216 for the healthcare-specific blockchain system performed by the adjudication node 202 includes domain-specific computational challenges, which can include the system presenting domain-specific computational challenges that adjudication node(s) 202 must solve to validate transactions and create new blocks in the blockchain. These challenges may involve analyzing complex healthcare data, simulating treatment scenarios, or optimizing resource allocation based on value delivered.

In various embodiments, the proof-of-work 216 for the healthcare-specific blockchain system performed by the adjudication node 202 includes collaboration with industry experts (e.g., a human in the loop 220), which can include incorporating in the proof-of-work mechanism input from healthcare professionals and experts to ensure that the evaluation metrics and computational challenges remain relevant and up-to-date with the latest developments in the healthcare industry.

In various embodiments, the business level proof-of-work principle 216 for the healthcare-specific blockchain 208 operates by sending data on healthcare transactions 204 associated with value-based agreements 206 via smart contracts 212 to the adjudication node(s) 202. The adjudication node 202 evaluates these transactions based on the predefined evaluation metrics in the evaluation metrics repository 214 and based on the AI-driven insights generated from real-world healthcare parameters. The adjudication node 202 solves domain-specific computational challenges related to the value delivered in the healthcare services. Upon successfully solving the challenges, the adjudication node 202 validates the transactions, generates a new block using the healthcare-specific proof-of-work principle, and appends the new block to the stakeholder's smart contract blockchain. By incorporating a healthcare-specific business level proof-of-work principle, the value-based healthcare adjudication system maintains the integrity and security of the blockchain while ensuring that the evaluation and compensation of healthcare services remain focused on delivering value and improving patient outcomes.

Referring again to the adjudication node 202, the adjudication node 202 is configured to solve domain-specific challenges by leveraging advanced technologies and methodologies that are tailored to the unique requirements of the healthcare industry. The process of solving domain-specific challenges at the adjudication node 202 involves data collection and integration in which the adjudication node 202 gathers relevant healthcare data from various sources, such as electronic health records (EHRs), medical imaging systems, and IoT devices. The adjudication node 202 then integrates this data, ensuring consistency and accuracy. In various embodiments, the adjudication node can perform data feature extraction and preprocessing in which the adjudication node 202 identifies and extracts relevant features from the collected data, focusing on those that are crucial for solving the domain-specific challenges. These features may include patient demographics, diagnostic information, treatment plans, and clinical outcomes. The adjudication node 202 preprocesses the data to ensure that it is in a suitable format for further analysis.

In various embodiments, the adjudication node 202 is also configured to perform domain-specific AI algorithms and machine learning techniques in which the adjudication node 202 employs algorithms and techniques, such as supervised learning, unsupervised learning, and reinforcement learning, to analyze the healthcare data and generate insights that are relevant to the domain-specific challenges. In various embodiments, these algorithms may involve one or more of predictive modeling to forecast patient outcomes and treatment success rates, classification algorithms to identify patterns and trends in healthcare data, clustering techniques to group patients with similar characteristics or conditions, optimization algorithms to determine the most cost-effective treatment options, and/or other algorithms and techniques.

In various embodiments, the adjudication node 202 is also configured to perform expert input and collaboration in which the adjudication node 202 collaborates with healthcare professionals and experts to ensure that the solutions generated by the AI algorithms and machine learning techniques are accurate, reliable, and aligned with the latest developments in the healthcare industry. This collaboration may involve incorporating expert feedback into the system or using expert knowledge to fine-tune the AI algorithms.

In various embodiments, the adjudication node 202 is also configured to perform iterative refinement and validation in which the adjudication node continuously refines the solutions to the domain-specific challenges by incorporating new data, updating the AI algorithms, and fine-tuning the machine learning models. The adjudication node 202 validates the solutions by comparing them against established benchmarks and performance metrics, ensuring that the solutions are accurate and reliable.

In various embodiments, the adjudication node 202 is also configured to perform decision-making and adjudication in which, once the adjudication node 202 has generated a solution to the domain-specific challenge, the adjudication node 202 uses this information to make a decision regarding the value of the healthcare transaction. This decision is based on the predefined evaluation metrics, the AI-driven insights, and the real-world healthcare parameters. The adjudication node then validates the transaction and generates a new block in the blockchain using the healthcare-specific proof-of-work principle.

Although FIG. 2 illustrates one example of adjudication system architecture 200, various changes may be made to FIG. 2. For example, various components and functions in FIG. 2 may be combined, further subdivided, replicated, or rearranged according to particular needs. Also, one or more additional components and functions may be included if needed or desired.

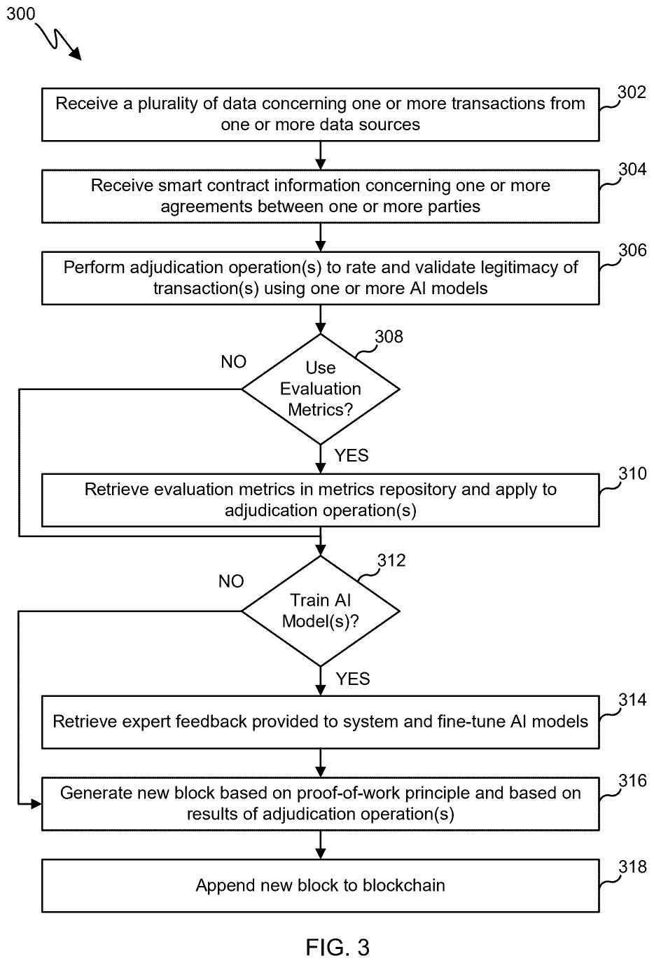

FIG. 3 illustrates an example method 300 for value based adjudication in accordance with this disclosure. For ease of explanation, the method 300 shown in FIG. 3 is described as being performed using the electronic device 101 in the network configuration 100 of FIG. 1. However, the method 300 could be performed using any other suitable device(s), such as the server 106, and in any other suitable system(s).

At step 302, at least one processing device, such as a processor of a secure adjudication device like the secure adjudication node 202 of FIG. 2, receives a plurality of data concerning one or more transactions, such as the transactions 204 of FIG. 2, from one or more data sources, such as the data sources 201, 203, 205, 207 of FIG. 2. The data sources may have their data stored in the form of databases. At step 304, the at least one processing device receives smart contract information concerning one or more agreements between one or more parties, such as the smart contracts 212 concerning the agreements 206 of FIG. 2.

In various embodiments, the secure adjudication device can be a decentralized node that is responsible for evaluating transactions based on the value delivered. The adjudication device can map the contractual nature of healthcare transactions to the agreed-upon value criteria and assigns ratings accordingly. In various embodiments, the transactions data is associated with an item of mutual agreement via the smart contracts. That is, the contracts represent value based agreements entered into between parties such as payors, provides, patients, etc. These agreements can be processed by a smart contract generator to create one or more smart contracts and data associated with the smart contracts and transactions are provided to the adjudication device, which evaluates the transactions based on the defined value criteria established by the parameters outlined in the smart contracts. In various embodiments, the item of mutual agreement constitutes a defined value delivered in the context of a care activity that uses a pharmaceutical drug. The set of healthcare transactions that define value could be a combined with a set of healthcare parameters (e.g., patient diagnosis, disease state, treatment protocols, Rx drugs, clinical evidence, outcomes, etc.).

At step 306, the at least one processing device performs adjudication operation(s) to rate and validate the legitimacy of the transaction(s) using one or more AI models. The adjudication device employs AI algorithms and machine learning techniques to analyze the transactions and assign ratings accordingly. These advanced AI algorithms and machine learning techniques are employed by the adjudication device to analyze healthcare transactions, extract relevant data, and optimize the adjudication process to assist with identifying patterns and trends in healthcare parameters, predicting patient outcomes, and determining the efficacy of treatments. The adjudication device can also map the contractual nature of healthcare transactions associated with a stakeholder to the contractual value agreement. The adjudication device can thus award criteria ratings for the transactions.

At step 308, it is determined whether evaluation metrics are to be used in performing the adjudication operation(s). If not, the method 300 moves to step 312. However, if so, at step 310, the at least one processing device retrieves evaluation metrics in a metrics repository, such as the repository 214 of FIG. 2, and applies the metrics to the adjudication operation(s).

At step 312, it is determined whether to train the AI models, which can be in response to receiving expert feedback provided to the system. If not, the method 300 moves to step 316. If so, at step 314, the at least one processing devices retrieves the expert feedback and fine-tunes at least one of the AI models based on the expert feedback. In some embodiments, the AI models can be fine-tuned prior to completing the adjudication operation(s) of step 306.

The adjudication device can be configured to solve domain-specific challenges by leveraging advanced technologies and methodologies that are tailored to the unique requirements of the healthcare industry. The process of solving domain-specific challenges at the adjudication device involves data collection and integration in which the adjudication device gathers relevant healthcare data from various sources, such as electronic health records (EHRs), medical imaging systems, and IoT devices. The adjudication device then integrates this data, ensuring consistency and accuracy. In various embodiments, the adjudication device can perform data feature extraction and preprocessing in which the adjudication device identifies and extracts relevant features from the collected data, focusing on those that are crucial for solving the domain-specific challenges. These features may include patient demographics, diagnostic information, treatment plans, and clinical outcomes. The adjudication device preprocesses the data to ensure that it is in a suitable format for further analysis.

In various embodiments, the adjudication device is also configured to perform domain-specific AI algorithms and machine learning techniques in which the adjudication device employs algorithms and techniques, such as supervised learning, unsupervised learning, and reinforcement learning, to analyze the healthcare data and generate insights that are relevant to the domain-specific challenges. In various embodiments, these algorithms may involve one or more of predictive modeling to forecast patient outcomes and treatment success rates, classification algorithms to identify patterns and trends in healthcare data, clustering techniques to group patients with similar characteristics or conditions, optimization algorithms to determine the most cost-effective treatment options, and/or other algorithms and techniques.

In various embodiments, the adjudication device is also configured to perform expert input and collaboration in which the adjudication device collaborates with healthcare professionals and experts to ensure that the solutions generated by the AI algorithms and machine learning techniques are accurate, reliable, and aligned with the latest developments in the healthcare industry. This collaboration may involve incorporating expert feedback into the system or using expert knowledge to fine-tune the AI algorithms.

In various embodiments, the adjudication device is also configured to perform iterative refinement and validation in which the adjudication device continuously refines the solutions to the domain-specific challenges by incorporating new data, updating the AI algorithms, and fine-tuning the machine learning models. The adjudication device validates the solutions by comparing them against established benchmarks and performance metrics, ensuring that the solutions are accurate and reliable.

In various embodiments, the adjudication device is also configured to perform decision-making and adjudication in which, once the adjudication device has generated a solution to the domain-specific challenge, the adjudication device uses this information to make a decision regarding the value of the healthcare transaction. This decision is based on the predefined evaluation metrics, the AI-driven insights, and the real-world healthcare parameters. The adjudication device then validates the transaction and generates a new block in the blockchain using the healthcare-specific proof-of-work principle.

At step 316, the at least one processing device generates a new block based on proof-of-work principle and based on results of adjudication operation(s). At step 318, the at least one processing device causes the new block to be appended to a blockchain, such as a blockchain of the blockchain network 208 of FIG. 2. As noted above, the adjudication device assigns ratings to the transactions and validates their legitimacy. Upon establishing the validity of the transactions, the adjudication device generates a new block using a proof-of-work principle. The proof-of-work principle is a consensus mechanism used to validate healthcare transactions and create new blocks in the blockchain. The proof-of-work principle uses devices in the network to solve complex computational problems, preventing fraud and maintaining the integrity of the system. The proof-of-work principle 216 is a healthcare-specific business level proof-of-work principle that is incorporated into the blockchain system/network 208 for value-based healthcare transaction adjudication. This proof-of-work mechanism can thus focus on evaluating the value delivered in healthcare services, leveraging domain-specific knowledge, AI-driven insights, and real-world healthcare parameters to validate transactions and maintain the integrity of the system. In various embodiments, the proof-of-work for the healthcare-specific blockchain system can be performed by the adjudication device includes domain-specific evaluation metrics (which can be included in the evaluation metrics repository), which can utilize a set of predefined, healthcare-specific evaluation metrics that reflect the value delivered by healthcare services. These metrics may include patient satisfaction, treatment success rates, cost-effectiveness, and adherence to best practice guidelines.

Although FIG. 3 illustrates one example of a method 300 for value based adjudication various changes may be made to FIG. 3. For example, while shown as a series of steps, various steps in FIG. 3 could overlap, occur in parallel, occur in a different order, or occur any number of times (including zero times).

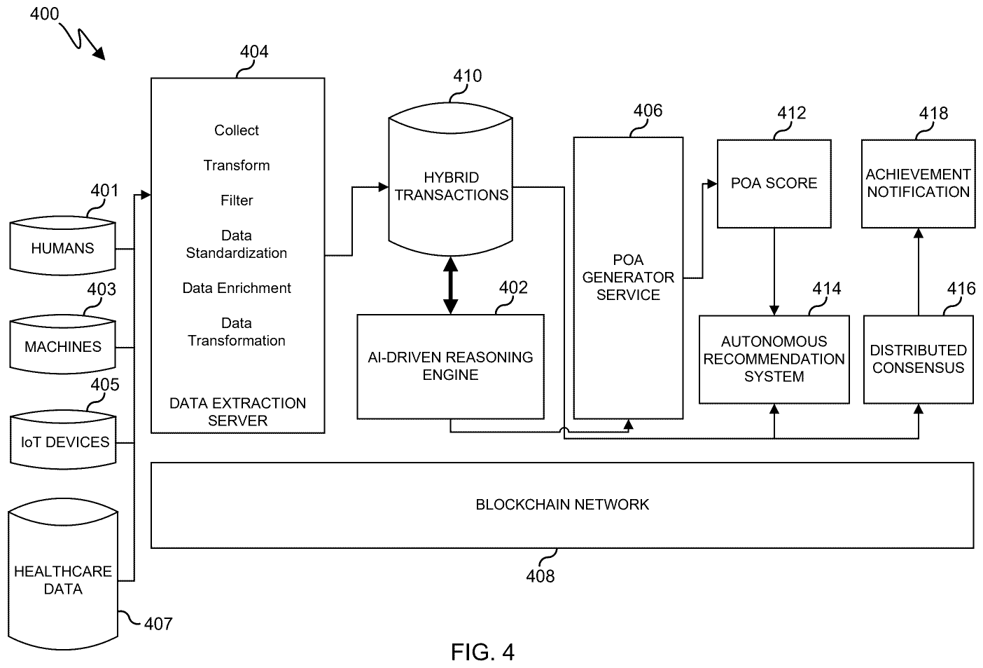

FIG. 4 illustrates an example hybrid proof of achievement architecture 400 in accordance with this disclosure. For ease of explanation, the architecture 400 shown in FIG. 4 is described as being implemented on or supported by the electronic device 101 in the network configuration 100 of FIG. 1. However, the architecture 400 shown in FIG. 4 could be used with any other suitable device(s) and in any other suitable system(s), such as when the architecture 400 is implemented on or supported by the server 106.

Distributed consensus algorithms form a cornerstone of blockchains. To build a good ecology of participants, autonomous recommendation of agreement is an important aspect of blockchain systems. Current enterprise solutions proof of achievement (POA) comes as a statistical output lacking an empirical nature. POA is aimed at arriving at a consensus by all interested parties exchanging the relevant activities. The architecture 400 provides an AI-powered distributed consensus system for hybrid (human and machine) transactions, focusing on rapidity, irreversibility, and agreement. This system aims to enhance the traditional POA approach by incorporating advanced AI techniques and enabling autonomous recommendation of agreement to foster a robust ecosystem of participants. The unique POA system of this disclosure involves all interested parties exchanging relevant activities, while a sophisticated reasoning engine facilitates the generation of a POA score, signifying relative achievement with respect to the original agreement or contract.

As shown in FIG. 4, the architecture 400 includes an AI driven reasoning engine 402, a data extraction server 404, a POA generator service 406, and a blockchain network 408. The data extraction server 404 can be connected to various data sources including human sources 401, machine sources 403, IoT sources 405, and healthcare data sources 407. The data sources may have their data stored in the form of databases. The data extraction server 404 can be a dedicated server responsible for extracting relevant data from various sources involved in hybrid transactions. The data extraction server supports seamless integration with human and machine participants, such as smart contracts, IoT devices, and automated systems, to collect and preprocess data required for the AI-driven reasoning engine. The data extraction server 404, using the various data sources, collects, transforms and/or filters the data, and/or performs data standardization, data enrichment, and/or data transformation on the data. For example, the data extraction server performs data acquisition and normalization by collecting data from the multiple sources and performs data preprocessing techniques such as data cleaning, transformation, and normalization to ensure a consistent format and representation across different sources.

In various embodiments, data collection performed by the data extraction server 404 involves the data extraction server 404 connecting to various data sources, such as databases, APIs, IoT devices, and third-party services, to collect the necessary data related to the hybrid transactions. In various embodiments, data transformation performed by the data extraction server 404 involves the data extraction server 404 transforming the collected data into a unified format compatible with the AI-driven reasoning engine 402. This can include parsing, decoding, and converting data to ensure seamless integration with the rest of the system.

In various embodiments, data filtering performed by the data extraction server 404 involves the data extraction server 404 filtering the collected data based on predefined criteria or rules, ensuring that only relevant and high-quality data is passed on to the AI-driven reasoning engine for further analysis. In various embodiments, data enrichment performed by the data extraction server 404 involves the data extraction server 404 enriching the collected data with additional information from external sources, if required, to enhance the context and accuracy of the data fed into the AI-driven reasoning engine. In various embodiments, data transmission performed by the data extraction server 404 involves the data extraction server 404 securely transmitting the preprocessed data to the AI-driven reasoning engine 402 for further analysis, feature extraction, and POA score calculation.

The processed data can be securely stored on the blockchain network 408, ensuring data integrity, immutability, and traceability. The preprocessed data can be stored in or used by a hybrid transactions data store or hybrid transactions operation 410. The hybrid transactions are transactions involving both human and machine participants, such as smart contracts, IoT devices, and automated systems, in a blockchain-based ecosystem.

The AI-driven reasoning engine 402 is an advanced AI component of the architecture 400 that processes the data from the relevant activities exchanged by the interested parties. The AI-driven reasoning engine 402 employs machine learning algorithms, natural language processing, and pattern recognition techniques to analyze the data and derive meaningful insights and facilitate the consensus process in a transparent and explainable manner. The AI-driven reasoning engine 402 is responsible for processing and analyzing the data from relevant activities exchanged by interested parties, ultimately generating a POA score for each participant.

In various embodiments, the reasoning engine 402 can perform data preprocessing such as employing data preprocessing techniques to clean, normalize, and standardize the data from relevant activities, ensuring consistency and compatibility across different formats and sources, and improving the accuracy and efficiency of subsequent analysis.

In various embodiments, additionally or alternatively, the reasoning engine 402 can perform feature extraction by using natural language processing and pattern recognition techniques, to identify and extract relevant features and variables from the preprocessed data. These features represent aspects of the relevant activities and serve as input for machine learning operations.

In various embodiments, additionally or alternatively, the reasoning engine 402 can perform machine learning operations such that the reasoning engine leverages various machine learning techniques, such as supervised learning, unsupervised learning, and reinforcement learning, to analyze the extracted features and identify patterns, correlations, and trends. These algorithms adapt and improve over time as new data is introduced, enhancing the overall performance of the reasoning engine.

In various embodiments, additionally or alternatively, the reasoning engine 402 can perform knowledge representation and reasoning operations in which the reasoning engine 402 utilizes knowledge representation techniques, such as ontologies, semantic networks, and rule-based systems, to store and organize the acquired knowledge. This structured knowledge base enables the engine 402 to reason and infer new insights based on the existing information.

In various embodiments, additionally or alternatively, the reasoning engine 402 can perform, such as in conjunction with the POA generator service 406, a POA score calculation. The AI-driven reasoning engine 402 and the POA generation service 406 can be seamlessly integrated into the blockchain network 408, ensuring secure and transparent record-keeping of hybrid transactions and the resulting POA scores. The reasoning engine 402 computes a POA score 412 for each participant by evaluating their performance concerning the original agreement or contract. The calculation takes into account various factors, such as the relevance, quality, and timeliness of the activities exchanged, as well as any predefined performance metrics or benchmarks. The POA generator service 406 can be a specialized service responsible for calculating and assigning POA scores to the participants of hybrid transactions. The POA generator service 406 interacts with the AI-driven reasoning engine 402, leveraging its analytical capabilities and knowledge base to derive meaningful insights and generate accurate POA scores for each participant.

In various embodiments, the POA generator service 406 can perform POA score request handling in which the service receives and processes requests for POA score calculation from the participants of hybrid transactions or the system itself. In various embodiments, the POA generator service 406 can interact with the AI-driven reasoning engine 402, such as by communicating with the AI-driven reasoning engine 402 and requesting analysis, insights, and evaluations required for POA score calculation.

In various embodiments, the POA generator service 406 can perform at least a part of the POA score calculation based on the information received from the AI-driven reasoning engine 402, in which the POA generator service 406 computes the POA score 412 for each participant, taking into account various factors such as relevance, quality, timeliness of the activities exchanged, and any predefined performance metrics or benchmarks.

In various embodiments, the POA generator service 406 can perform POA score assignment in which the POA generator service 406 assigns the calculated POA scores to the respective participants, updating their records within the blockchain network 408 and a distributed consensus operation 416.

In various embodiments, the POA generator service 406 can perform POA score reporting in which the POA generator service 406 generates and delivers reports on the POA scores 412 to the relevant parties, such as participants, regulators, and system administrators. The reports provide insights into the participants' performance and achievement in fulfilling the original agreement or contract, promoting transparency and accountability within the hybrid transaction ecosystem.

In various embodiments, the POA generator service 406 can perform continuous improvement in which the POA generation service 406 monitors and evaluates the accuracy and effectiveness of the POA scores 412, collecting feedback from the parties and providing it to the AI-driven reasoning engine for further refinement and improvement of the operations and knowledge base of the architecture 400.

In various embodiments, additionally or alternatively, the reasoning engine 402 can perform continuous learning and improvement operations in which the AI-driven reasoning engine 402 continuously learns from new data and feedback, refining its machine learning models and updating the knowledge base to ensure the most accurate and up-to-date POA scores. This dynamic learning process enables the system to adapt to evolving requirements and conditions in the hybrid transaction ecosystem.

In various embodiments, additionally or alternatively, the reasoning engine 402 can provide explainability and interpretability. That is, the reasoning engine 402 is designed to provide explainable and interpretable results, allowing parties to understand the rationale behind the generated POA scores. This transparency is crucial for building trust and confidence in the system, as well as facilitating the consensus process among participants.

In various embodiments, the POA generator service 406 works with the AI-driven reasoning engine to calculate the POA score 412 for each participant, representing their relative achievement in fulfilling the terms and conditions of the original agreement or contract, which is passed to an autonomous recommendation system 414. The autonomous recommendation system 414 is an AI-powered mechanism that autonomously recommends agreements based on the POA scores, fostering a self-sustaining ecosystem of participants and encouraging continuous improvement in performance. The architecture 400 also includes the distributed consensus operation 416 that uses a decentralized protocol that leverages the AI-driven reasoning engine and the POA scores to achieve consensus among the participants in the hybrid transactions. An achievement notification 418 can then be transmitted to one or more parties.

Although FIG. 4 illustrates one example of a hybrid proof of achievement architecture 400, various changes may be made to FIG. 4. For example, various components and functions in FIG. 4 may be combined, further subdivided, replicated, or rearranged according to particular needs. Also, one or more additional components and functions may be included if needed or desired.

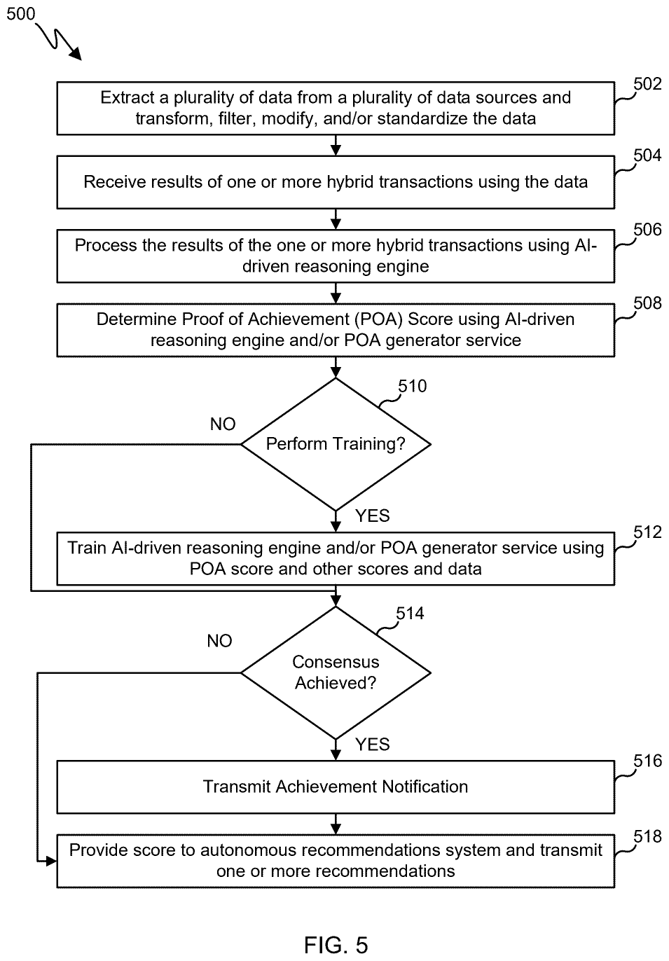

FIG. 5 illustrates an example method 500 for hybrid proof of achievement in accordance with this disclosure. For ease of explanation, the method 500 shown in FIG. 5 is described as being performed using the electronic device 101 in the network configuration 100 of FIG. 1. However, the method 500 could be performed using any other suitable device(s), such as the server 106, and in any other suitable system(s).

At step 502, at least one processing device extracts a plurality of data from a plurality of data sources and transforms, filters, modifies, and/or standardizes the data. For example, as described with respect to FIG. 4, the data extraction server 404 can be used to extract the data from sources such as the sources 401, 403, 405, 407 and perform the manipulation of the data. At step 504, the at least one processing device receives results of one or more hybrid (i.e., human and machine) transactions using the data.

At step 506, the at least one processing device processes the results of the one or more hybrid transactions are processed using an AI-driven reasoning engine, such as the engine 402. As also described with respect to FIG. 4, the AI-driven reasoning engine 402 is an advanced AI component that processes the data from the relevant activities exchanged by interested parties. The AI-driven reasoning engine employs machine learning algorithms, natural language processing, and pattern recognition techniques to analyze the data and derive meaningful insights and facilitate the consensus process in a transparent and explainable manner. In various embodiments, the reasoning engine can perform data preprocessing such as employing data preprocessing techniques to clean, normalize, and standardize the data from relevant activities, ensuring consistency and compatibility across different formats and sources, and improving the accuracy and efficiency of subsequent analysis.

In various embodiments, additionally or alternatively, the reasoning engine can perform feature extraction by using natural language processing and pattern recognition techniques, to identify and extract relevant features and variables from the preprocessed data. These features represent aspects of the relevant activities and serve as input for machine learning operations. In various embodiments, additionally or alternatively, the reasoning engine can perform knowledge representation and reasoning operations in which the reasoning engine utilizes knowledge representation techniques, such as ontologies, semantic networks, and rule-based systems, to store and organize the acquired knowledge. This structured knowledge base enables the engine to reason and infer new insights based on the existing information.

At step 508, the at least one processing device determines a POA score using the AI-driven reasoning engine and/or a POA generator service, such as the POA generator service 406. The reasoning engine is responsible for processing and analyzing the data from relevant activities exchanged by interested parties, ultimately generating a POA score for each participant. In some embodiments, the POA generator service can be part of the AI-driven reasoning engine. The AI-driven reasoning engine and the POA generation service can be seamlessly integrated into a blockchain network, such as the blockchain network 408, ensuring secure and transparent record-keeping of hybrid transactions and the resulting POA scores. The reasoning engine computes a POA score for each participant by evaluating their performance concerning the original agreement or contract. The calculation takes into account various factors, such as the relevance, quality, and timeliness of the activities exchanged, as well as any predefined performance metrics or benchmarks. The POA generator service can be a specialized service responsible for calculating and assigning POA scores to the participants of hybrid transactions. The POA generator service can interact with the AI-driven reasoning engine, leveraging its analytical capabilities and knowledge base to derive meaningful insights and generate accurate POA scores for each participant.

At step 510, it is determined whether to train the reasoning engine and/or the POA generator service. For example, in various embodiments, the reasoning engine can perform machine learning operations such that the reasoning engine leverages various machine learning techniques, such as supervised learning, unsupervised learning, and reinforcement learning, to analyze the extracted features and identify patterns, correlations, and trends. These algorithms can be adapted and improved over time via training as new data is introduced, enhancing the overall performance of the reasoning engine. In various embodiments, the POA generator service can perform continuous improvement in which the POA generation service monitors and evaluates the accuracy and effectiveness of the POA scores, collecting feedback from the parties and providing it to the AI-driven reasoning engine for further refinement and improvement of system operations and knowledge bases.

If, at step 510, it is determined that training is not to be performed, the method 500 moves to step 514. If, at step 510, it is determined that training is to be performed, the method 500 moves to step 512, at which the reasoning engine and/or the POA generator service are trained using the POA score generated at step 508, and other scores and data, such as ground truth data received at the system via a training feedback loop. The method 500 then moves to step 514.

At step 514, it is determined whether consensus is achieved. That is, the method can include obtaining a distributed consensus, such as via distributed consensus operation 416, that uses a decentralized protocol that leverages the AI-driven reasoning engine and the POA scores to achieve consensus among the participants in the hybrid transactions. If consensus is not achieved, the method 500 moves to step 518. If consensus is achieved, then, at step 516, in various embodiments, an achievement notification can be transmitted to one or more parties. At step 518, the at least one processing device causes the score to be provided to an autonomous recommendations system (e.g., the autonomous recommendations system 414) which transmits one or more recommendations. For example, the autonomous recommendation system is an AI-powered mechanism that autonomously recommends agreements based on the POA scores, fostering a self-sustaining ecosystem of participants and encouraging continuous improvement in performance.

Although FIG. 5 illustrates one example of a method 500 for hybrid proof of achievement, various changes may be made to FIG. 5. For example, while shown as a series of steps, various steps in FIG. 5 could overlap, occur in parallel, occur in a different order, or occur any number of times (including zero times).

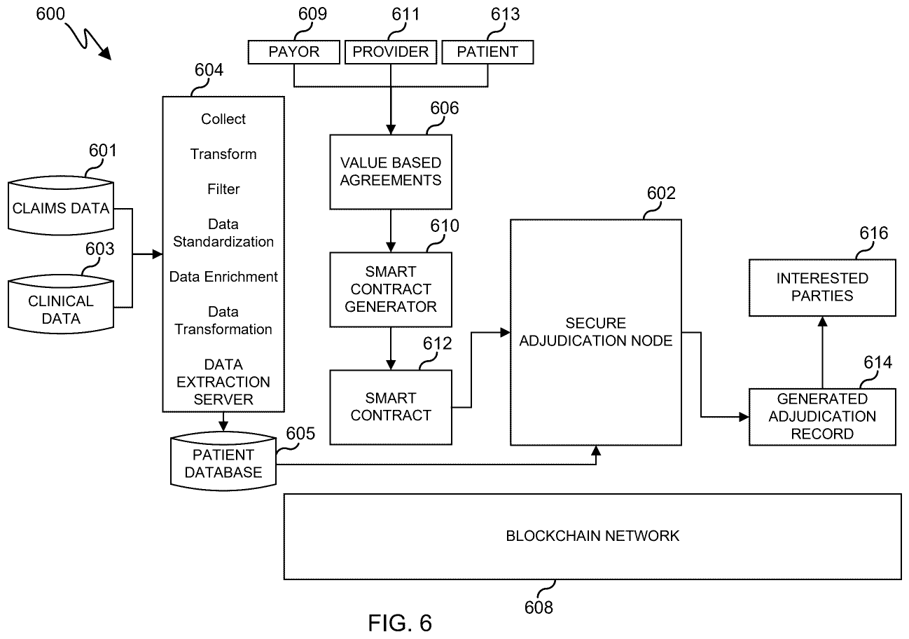

FIG. 6 illustrates an example validation and adjudication architecture 600 in accordance with this disclosure. For ease of explanation, the architecture 600 shown in FIG. 6 is described as being implemented on or supported by the electronic device 101 in the network configuration 100 of FIG. 1. However, the architecture 600 shown in FIG. 6 could be used with any other suitable device(s) and in any other suitable system(s), such as when the architecture 600 is implemented on or supported by the server 106.

As shown in FIG. 6, the architecture 600 leverages healthcare transactions data and computes values for adjudication via a proof-of-work (POW) approach. As shown in FIG. 6, the architecture 600 includes a secure adjudication node or device 602 and a blockchain network 608. The secure adjudication node 602 can be provided data derived from various data sources involved in healthcare transactions, such as healthcare claims data 601 and/or clinical data 603. The data sources may have their data stored in the form of databases.

As shown in FIG. 6, the architecture 600 also includes a data extraction server 604. The data extraction server 604 can be connected to various data sources including the claims data 601 and/or the clinical data 603. The data sources may have their data stored in the form of databases. The data extraction server 604 can be a dedicated server responsible for extracting relevant data from various sources. The data extraction server supports seamless integration with human and machine participants, such as smart contracts, IoT devices, and automated systems, to collect and preprocess data used by the secure adjudication node 602. The data extraction server 604, using the various data sources, collects, transforms and/or filters the data, and/or performs data standardization, data enrichment, and/or data transformation on the data. For example, the data extraction server performs data acquisition and normalization by collecting data from the multiple sources and performs data preprocessing techniques such as data cleaning, transformation, and normalization to ensure a consistent format and representation across different sources.

In various embodiments, data collection performed by the data extraction server 604 involves the data extraction server 604 connecting to various data sources, such as databases, APIs, IoT devices, and third-party services, to collect the necessary data. In various embodiments, data transformation performed by the data extraction server 604 involves the data extraction server 604 transforming the collected data into a unified format compatible with the adjudication node 602. This can include parsing, decoding, and converting data to ensure seamless integration with the rest of the system.

In various embodiments, data filtering performed by the data extraction server 604 involves the data extraction server 604 filtering the collected data based on predefined criteria or rules, ensuring that only relevant and high-quality data is passed on to the adjudication node 602 for further analysis. In various embodiments. data enrichment performed by the data extraction server 604 involves the data extraction server 604 enriching the collected data with additional information from external sources, if required, to enhance the context and accuracy of the data fed into the adjudication node 602. In various embodiments, data transmission performed by the data extraction server 604 involves the data extraction server 604 securely transmitting the preprocessed data to the secure adjudication node 602 for further analysis and/or storing the information is a separate database that can be accessed by the adjudication node 602, such as a patient database 605. In various embodiments, the processed data can be also securely stored on the blockchain network 608, ensuring data integrity, immutability, and traceability.

The secure adjudication node 602, in various embodiments, is a decentralized node within the blockchain network 608 that is responsible for evaluating healthcare transactions based on the value delivered. The adjudication node maps the contractual nature of healthcare transactions to the agreed-upon value criteria and assigns ratings accordingly.

The architecture 600 is configured to receive information on healthcare transactions associated with an item of mutual agreement via smart contracts. That is, the contracts can represent value based agreements 606 entered into between at least one payor 209, at least one provider 611, and at least one patient 613. These agreements 606 are then processed by a smart contract generator 610 to create one or more smart contracts 612. Value based healthcare transactions associated with an item of mutual agreement via smart contracts are sent to an adjudication node 602. This can include the adjudication node(s) 602 receiving data on the healthcare transactions that includes a set of healthcare tokens that represent healthcare actions taken with respect to a stakeholder. For example, the healthcare tokens might include test results for a patient and a corresponding diagnosis from a doctor. The adjudication node 602 can then obtain a contractual block identifier of the involved parties' agreement of value. The agreement represents a measure of healthcare outcome in the form of a programmatic representation of delivered value. In some embodiments, the adjudication node 602 can also receive a validity requirement with respect to the healthcare actions indicating criteria that to be met in order for the system to accept an adjudication event with respect to the transaction. The adjudication device 602 can also adjudicate the healthcare actions by obtaining a digital signature of a validator.

Once the various pieces of information have been collected, the adjudication device 602 calculates a validity block based on the transaction and according to the validity requirements as a function of the healthcare action parameters including the security token, agreement identifier, the set of healthcare tokens, and the digital signature. Should the requirements be met, the adjudication device 602 can cause the smart contract 212 to be updated, possibly by appending the block to the chain.

In some embodiments, such as described with respect to FIG. 23, the adjudication node 602 can assign ratings to the transactions and validate their legitimacy, which can include using AI models and machine learning techniques. Upon establishing the validity of the transactions, the adjudication node 602 can generate a new block using a proof-of-work principle and append the block to the blockchain of the blockchain network 608. As shown in FIG. 6, the secured adjudication node 602 can also generate an adjudication record 614 that can be provided to interested parties 616.

Although FIG. 6 illustrates one example of a validation and adjudication architecture 600, various changes may be made to FIG. 6. For example, various components and functions in FIG. 6 may be combined, further subdivided, replicated, or rearranged according to particular needs. Also, one or more additional components and functions may be included if needed or desired.

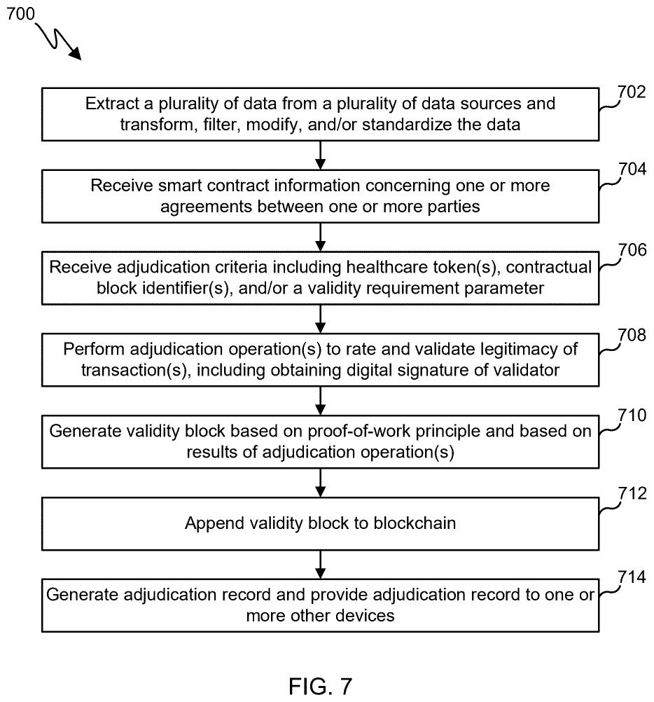

FIG. 7 illustrates an example validation and adjudication method 700 in accordance with this disclosure. For ease of explanation, the method 700 shown in FIG. 7 is described as being performed using the electronic device 101 in the network configuration 100 of FIG. 1. However, the method 700 could be performed using any other suitable device(s), such as the server 106, and in any other suitable system(s).

At step 702, at least one processing device extracts a plurality of data from a plurality of data sources and transforms, filters, modifies, and/or standardizes the data. For example, as described with respect to FIG. 6, the data extraction server 604 can be used to extract the data from sources such as the sources 401, 403 and perform the manipulation of the data. At step 704, the at least one processing device receives smart contract information concerning one or more agreements between one or more parties. For example, the at least one processing device can be included in a decentralized node such as the adjudication node 602 of FIG. 6, that is responsible for evaluating healthcare transactions based on the value delivered. The adjudication node maps the contractual nature of healthcare transactions to the agreed-upon value criteria and assigns ratings accordingly. That is, decentralized node is configured to receive information on healthcare transactions associated with an item of mutual agreement via smart contracts.

At step 706, the at least one processing device receives adjudication criteria such as a healthcare token(s), a contractual block identifier(s), and/or a validity requirement. This can include the adjudication node(s) receiving data on the healthcare transactions that includes a set of healthcare tokens that represent healthcare actions taken with respect to a stakeholder. For example, the healthcare tokens might include test results for a patient and a corresponding diagnosis from a doctor. The adjudication node can then obtain a contractual block identifier of the involved parties' agreement of value. The agreement represents a measure of healthcare outcome in the form of a programmatic representation of delivered value. In some embodiments, the adjudication node can also receive a validity requirement with respect to the healthcare actions indicating criteria that to be met in order for the system to accept an adjudication event with respect to the transaction.

At step 708, the at least one processing device performs adjudication operation(s) to rate and validate the legitimacy of the transaction(s). In some embodiments, this can include obtaining a digital signature of a validator. In some embodiments, the adjudication device employs AI algorithms and machine learning techniques to analyze the transactions and assign ratings accordingly. As part of step 708, the adjudication device maps the contractual nature of healthcare transactions associated with a stakeholder to the contractual value agreement. The adjudication device can thus award criteria ratings for the transactions.

At step 710, the at least one processing device, such as via the adjudication node, generates a validity block based on the transaction and according to the validity requirements as a function of the healthcare action parameters including the security token, agreement identifier, the set of healthcare tokens, and the digital signature. At step 712, the validity block is appended to the blockchain. At step 714, based on the results of the adjudication, an adjudication record can be generated and provided to one or more other electronic devices, such as electronic devices associated with the interested parties 616.

Although FIG. 7 illustrates one example of an adjudication method 700, various changes may be made to FIG. 7. For example, while shown as a series of steps, various steps in FIG. 7 could overlap, occur in parallel, occur in a different order, or occur any number of times (including zero times).

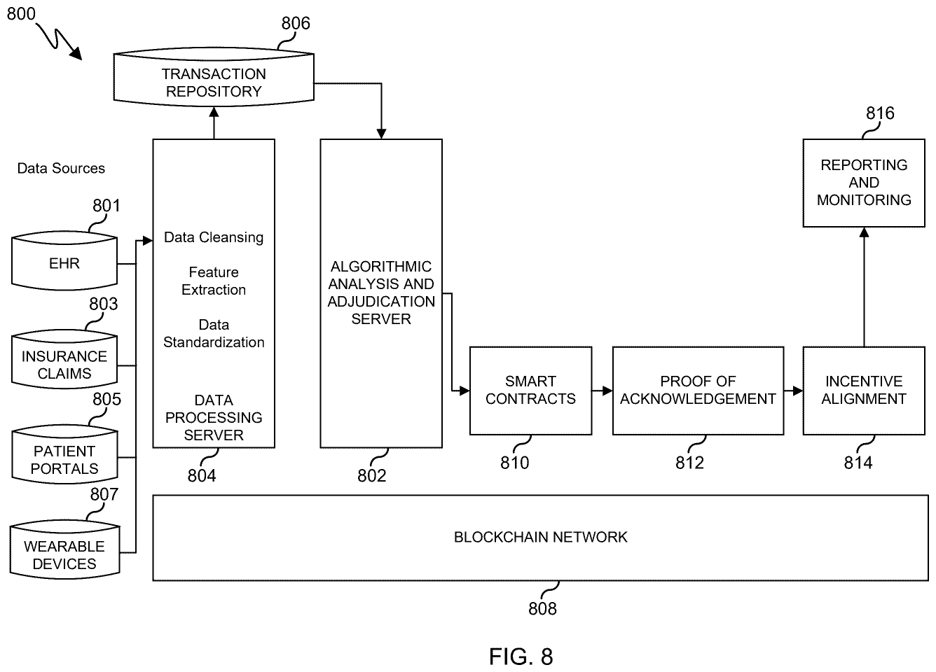

FIG. 8 illustrates an example proof of acknowledgement architecture 800 in accordance with this disclosure. For ease of explanation, the architecture 800 shown in FIG. 8 is described as being implemented on or supported by the electronic device 101 in the network configuration 100 of FIG. 1. However, the architecture 800 shown in FIG. 8 could be used with any other suitable device(s) and in any other suitable system(s), such as when the architecture 800 is implemented on or supported by the server 106.

The architecture 800 enables performance of distributed consensus and monitoring methods for evaluating needs and providing proof of acknowledgements. For example, the distributed consensus and monitoring methods can evaluate healthcare needs by combining patient health, financial, and engagement data addressing frictions in healthcare value-based care by using a distributed consensus and monitoring method that uses the combined patient health, financial, and engagement data. The architecture 800 also utilizes advanced AI techniques, blockchain technology, and a proof of acknowledgement (PoA) mechanism, to reduce friction, enhance efficiency, and promote transparency.

As shown in FIG. 8, various data sources can be accessed by a data processing server(s) 804 that manipulates the data for use by other components of the architecture 800, such as an adjudication server 802. The various data sources can include as electronic health record (EHR) sources 801, insurance claims sources 803, patient portal sources 805, wearable device sources 807, and/or other data sources. The data processing server can perform data cleansing, feature extraction, data standardization, and/or other operations using the data retrieved from the various data sources.

The adjudication server 802 is integrated into the system to automatically process transactions and resolve disputes or discrepancies. The adjudication server 802 leverages advanced AI algorithms to analyze transaction data, validate the legitimacy of claims, and calculate appropriate reimbursements based on predefined rules and conditions. By automating the adjudication process, the server helps to reduce the time and cost associated with manual claim reviews, enhancing overall efficiency and reducing financial friction. In some embodiments, the architecture 800 can include or be a part of the architectures 200, 400, and/or 600. For example, in some embodiments, the adjudication server 802 can include or be a part of the AI-driven event detection and notification system 211, the reasoning engine 402, and/or the reasoning engine 602.

The architecture 800 can also include a transaction repository 806. The transaction repository 806 is a secure, tamper-proof, repository for storing transactions data and relevant metadata. The transaction repository 806 utilizes blockchain technology supported by a blockchain network 808, and ensures the integrity and traceability of all transactions, enabling parties to review and audit activities with confidence. By leveraging use of the blockchain network 808, the system ensures secure, transparent, and tamper-proof storage of healthcare data. The decentralized nature of the blockchain network 808 promotes trust among parties and streamlines the exchange of information across various entities. The transaction repository 806 also facilitates real-time monitoring and analysis of transaction data, allowing parties to identify patterns, trends, and areas for improvement.

The adjudication server 802 can perform various algorithmic analysis and adjudication operations on the data stored in the transaction repository 806, which can include feature extraction data provided by the data processing server 804 that is formatted for use by one or more machine learning models executed by the adjudication server 802. The adjudication server employs advanced AI algorithms, such as machine learning and natural language processing, to analyze and interpret patient health, financial, and engagement data. This data-driven approach enables the identification of patterns and trends, allowing for more accurate predictions of healthcare needs and resource allocation. Advanced AI algorithms and machine learning techniques are employed to analyze the integrated data, uncovering patterns and trends related to healthcare costs, patient outcomes, and engagement levels.

This analysis helps identify areas of improvement and opportunities for optimizing resource allocation in value-based care. In various embodiments, the adjudication server 802 can also employ advanced AI techniques to identify potential fraudulent activities, such as duplicate billing, overcharging, or misrepresentation of services. By detecting and addressing fraudulent transactions early, the system helps to minimize financial losses and maintain the integrity of the healthcare financial ecosystem. By leveraging AI and machine learning, the architecture 800 enables data-driven decision-making related to financial resource allocation and utilization. This helps to identify inefficiencies, optimize processes, and prioritize value-based care initiatives, ultimately reducing financial friction and enhancing overall healthcare outcomes.

The architecture 800 uses the blockchain network 808 to securely store and share data among parties, ensuring data integrity and promoting trust and transparency. The blockchain network 808 uses a distributed consensus mechanism to assist in establishing agreement on the validity and prioritization of needs and resource allocations. The blockchain network 808 allows for data to be securely stored and shared among parties. For example, devices of the blockchain network 808 can represent healthcare providers, payers, and other involved parties.

The architecture 800 integrates smart contracts 810 to automate the execution of agreements and transactions based on predefined rules and conditions. This automation reduces the need for manual intervention, decreases transaction costs, and accelerates the overall processes of the architecture 800. In some embodiments, the smart contracts 810 can be used to automate billing and reimbursement processes based on predefined rules and conditions. This automation reduces manual intervention, decreases transaction costs, and accelerates the overall process, further reducing financial friction in the healthcare value-based care ecosystem.

The architecture 800 performs a proof of acknowledgement operation(s) 812 that acts as a consensus protocol to validate the accuracy and integrity of the healthcare data, thereby facilitating a trustworthy exchange of information. The proof of acknowledgement operation 812 minimizes the risk of data manipulation and ensures that all parties involved acknowledge the transactions. The proof of acknowledgement operation 812 validates the completion of specific actions or milestones in certain processes, such as healthcare processes, such as receiving a prescribed treatment, attending a follow-up appointment, or achieving a target health outcome. This validation serves as a basis for transactions and resource allocation decisions, ensuring that funds are directed towards value-based care initiatives that demonstrate tangible results.

The architecture 800 can also perform an incentive alignment operation(s) 814. For example, the incentive alignment operation 814 can link financial rewards and penalties to objective performance metrics and patient outcomes, which can, for example, help to align the incentives of healthcare providers, payers, and patients. This alignment encourages the adoption of evidence-based practices, promotes patient engagement, and fosters a culture of continuous improvement. The architecture 800 can incorporate dynamic incentive models that reward healthcare providers, patients, and other parties for their participation and adherence to value-based care principles. These incentives can be tailored to individual circumstances, ensuring that all parties are motivated to contribute to the improvement of healthcare outcomes.

The architecture 800 can also provide reporting and monitoring service(s) 816. The service 816 offers real-time monitoring and feedback capabilities, enabling healthcare providers to track and evaluate the effectiveness of their interventions, adjust their strategies accordingly, and enhance the overall quality of care. The architecture 800 is designed to integrate seamlessly with existing systems, such as existing healthcare information systems, ensuring smooth data exchange and reducing the friction associated with information silos. Among other benefits, the architecture 800 addresses various aspects of financial friction in healthcare value-based care, which refers to the inefficiencies, delays, and unnecessary costs associated with the allocation and distribution of financial resources in the healthcare sector. By streamlining financial transactions, promoting transparency, and enhancing collaboration among stakeholders, the system can reduce financial friction and optimize resource allocation.

The architecture 800 can integrate seamlessly with current healthcare financial transaction systems, such as electronic health records (EHRs), practice management systems, and billing platforms. This interoperability ensures smooth data exchange and minimizes the friction associated with information silos, allowing healthcare providers, payers, and other parties to collaborate more effectively. The architecture 800 also can support the development and implementation of customizable financial models that cater to the unique needs of various healthcare parties. These models can incorporate factors such as regional variations, population demographics, and specific healthcare objectives, ensuring that financial resources are allocated effectively and efficiently.

Although FIG. 8 illustrates one example of a proof of acknowledgement architecture 800, various changes may be made to FIG. 8. For example, various components and functions in FIG. 8 may be combined, further subdivided, replicated, or rearranged according to particular needs. Also, one or more additional components and functions may be included if needed or desired. For instance, the components 802, 804, 806, 808, 810, 812, 814, and 816 could all be a part of a same server system, such as being a part of one server or multiple servers in a distributed server architecture.

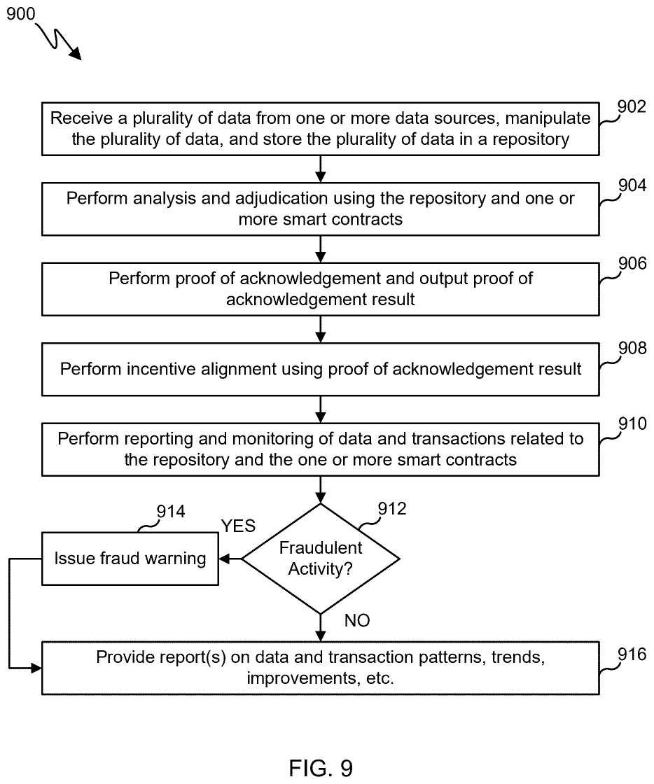

FIG. 9 illustrates an example proof of acknowledgement method 900 in accordance with this disclosure. For ease of explanation, the method 900 shown in FIG. 9 is described as being performed using the electronic device 101 in the network configuration 100 of FIG. 1. However, the method 900 could be performed using any other suitable device(s), such as the server 106, and in any other suitable system(s).

At step 902, a plurality of data is received from one or more data sources, the plurality of data is manipulated, and the plurality of data is stored in a repository. This can include the at least one processing device 120 retrieving the plurality of data from the plurality of data sources, such as data sources 801, 803, 805, 807, via a data processing server such as the data processing server 804. This can also include the processing device 120, such as via the data processing server, performing data cleansing, feature extraction, data standardization, and/or other operations using the data retrieved from the various data sources. This can also include the processing device 120 storing the manipulated data in the repository, such as the transaction repository 806.

At step 904, analysis and adjudication is performed using the repository and one or more smart contracts. This can include the at least one processing device 120 algorithmic analysis and adjudication operations on the data stored in the repository, such as via the adjudication server 802. This can also include the at least one processing device 120 consulting the one or more smart contracts, such as smart contracts 810, to automate the execution of agreements and transactions based on predefined rules and conditions and track patterns, trends, etc., and provide reporting on same or on other detected events such as based on fraudulent activity detection.

At step 906, proof of acknowledgement is performed and a proof of acknowledgement result is output. This can include the at least one processing device 120 executing the proof of acknowledgement operation(s) 812 to provide a consensus protocol to validate the accuracy and integrity of the healthcare data, thereby facilitating a trustworthy exchange of information. The proof of acknowledgement operation minimizes the risk of data manipulation and ensures that all parties involved acknowledge the transactions. The proof of acknowledgement operation validates the completion of specific actions or milestones in certain processes, such as healthcare processes, such as receiving a prescribed treatment, attending a follow-up appointment, or achieving a target health outcome. This validation serves as a basis for transactions and resource allocation decisions, ensuring that funds are directed towards value-based care initiatives that demonstrate tangible results.

At step 908, incentive alignment is performed using the proof of acknowledgement result. This can include the at least one processing device 120 executing the incentive alignment operation(s) 814 to link financial rewards and penalties to objective performance metrics and patient outcomes, which can, for example, help to align the incentives of healthcare providers, payers, and patients. This alignment encourages the adoption of evidence-based practices, promotes patient engagement, and fosters a culture of continuous improvement. This can also incorporate dynamic incentive models that reward healthcare providers, patients, and other parties for their participation and adherence to value-based care principles. These incentives can be tailored to individual circumstances, ensuring that all parties are motivated to contribute to the improvement of healthcare outcomes.

At step 910, reporting and monitoring of data and transactions related to the repository and the one or more smart contracts is performed. This can include the at least one processing device 120 executing the reporting and monitoring service(s) 816 to provide real-time monitoring and feedback capabilities, enabling the tracking and evaluation of the effectiveness of healthcare interventions, allowing for adjustment of strategies accordingly, and enhancing the overall quality of care.

At step 912, it is determined whether fraudulent activity is detected. This can include that at least one processing device 120, as part of executing the reporting and monitoring services, and in some embodiments also as part of performing the analysis and adjudication using the adjudication server, employing advanced AI techniques to identify potential fraudulent activities, such as duplicate billing, overcharging, or misrepresentation of services. By detecting and addressing fraudulent transactions early, the system helps to minimize financial losses and maintain the integrity of the healthcare financial ecosystem. Additionally, by leveraging AI and machine learning, the architecture enables data-driven decision-making related to financial resource allocation and utilization. This helps to identify inefficiencies, optimize processes, and prioritize value-based care initiatives, ultimately reducing financial friction and enhancing overall healthcare outcomes.

If, at step 912, no fraudulent activity is detected, the method 900 moves to step 916. If, at step 912, fraudulent activity is detected, at step 914, a fraud warning is issued. The fraud warning can be issued to various parties associated with the subject data and transactions to put them on notice of such possible fraudulent activities so that remedial action can be undertaken. The method then moves to step 916.

At step 916, one or more reports on data and transaction patterns, trends, improvements, etc., are provided. This can include the at least one processing device 120 executing the reporting and monitoring service(s) 816 to provide reports on patterns, trends, possible improvements, etc. related to the data, transactions, and smart contracts to one or more associated parties.

Although FIG. 9 illustrates one example of a proof of acknowledgement method 900, various changes may be made to FIG. 9. For example, while shown as a series of steps, various steps in FIG. 9 could overlap, occur in parallel, occur in a different order, or occur any number of times (including zero times).

It will be understood that the architectures 200, 400, 600, and 800 shown and described with respect to FIGS. 2, 4, 6, and 8 can be combined as part of one architecture in which the processes and methods facilitated by the architectures 200, 400, 600, and 800 can be performed by the one architecture. For example, the adjudication nodes 202, 602 could include the functionalities of one or more of the AI reasoning engine 402 or the algorithmic analysis and adjudication server 802 (e.g., the component can thus be an adjudication and reasoning engine), or vice versa. That is, the functionalities of each of the components 202, 402, 602, and 802 could be combined and performed by one device or by a one or more devices in cooperation.

Although this disclosure has been described with example embodiments, various changes and modifications may be suggested to one skilled in the art. It is intended that this disclosure encompass such changes and modifications as fall within the scope of the appended claims.

What is claimed is:

1. A system comprising:
at least one processing device configured to:
retrieve a plurality of data from one or more data sources;
perform, using an adjudication and reasoning engine, adjudication with respect to the plurality of data and using at least one machine learning model;
output adjudication results from the at least one machine learning model;
generate a validity block based on the adjudication results;
append the validity block to a blockchain;
receive results of one or more hybrid transactions stored in a transaction repository using the plurality of data;
determine, using the adjudication and reasoning engine, a proof of achievement score based on the results of the one or more hybrid transactions;
perform analysis and adjudication using the transaction repository and the at least one smart contract; and
determine and output a proof of acknowledgement result based on the analysis and adjudication.

2. The system of claim 1, wherein, to perform the adjudication, the at least one processing device is further configured to obtain a digital signature and associate the digital signature with the adjudication results.

3. The system of claim 1, wherein, to perform the adjudication, the at least one processing device is further configured to:
retrieve evaluation metrics from a metrics repository; and
apply the evaluation metrics to the adjudication to affect the adjudication results.

4. The system of claim 1, wherein the at least one processing device is further configured to:
perform a determination to train the at least one machine learning model based on expert feedback;
retrieve the expert feedback stored in the system; and
fine-tune the at least one machine learning model based on the retrieved expert feedback.

5. The system of claim 1, wherein the at least one processing device is further configured to:
manipulate the plurality of data by at least one of a transformation, a filter, a modification, and a standardization;
train the adjudication and reasoning engine using the proof of achievement score; and
provide based on the proof of achievement score, one or more recommendations.

6. The system of claim 1, wherein the at least one processing device is further configured to:
determine, based on the proof of achievement score, that a consensus is achieved; and
transmit an achievement notification indicating the achievement of the consensus.

7. The system of claim 1, wherein the plurality of data from the one or more data sources include smart contract information concerning agreements between one or more parties.

8. The system of claim 7, wherein the smart contract information includes one or more of a healthcare token, a contractual block identifier, or a validity requirement parameter.

9. The system of claim 1, wherein the at least one processing device is further configured to provide incentive alignment using the proof of acknowledgement result, including creating a link between a determined performance metric and one or more rewards and penalties.

10. The system of claim 1, wherein the at least one processing device is further configured to:
monitor data and transactions related to the transaction repository and at least one smart contract;
determine fraudulent activity is detected and issue a fraud warning to a controlling party in response; and
provide one or more reports on the data and transactions, wherein the one or more reports include at least one of patterns, trends, and improvements data.

11. A method comprising:
retrieving a plurality of data from one or more data sources;
performing, using an adjudication and reasoning engine, adjudication with respect to the plurality of data and using at least one machine learning model;
outputting adjudication results from the at least one machine learning model;
generating a validity block based on the adjudication results;
appending the validity block to a blockchain;
receiving results of one or more hybrid transactions stored in a transaction repository using the plurality of data;
determining, using the adjudication and reasoning engine, a proof of achievement score based on the results of the one or more hybrid transactions;
performing analysis and adjudication using the transaction repository and the at least one smart contract; and
determining and outputting a proof of acknowledgement result based on the analysis and adjudication.

12. The method of claim 11, wherein performing the adjudication includes obtaining a digital signature and associate the digital signature with the adjudication results.

13. The method of claim 11, wherein performing the adjudication includes:
retrieving evaluation metrics from a metrics repository; and
applying the evaluation metrics to the adjudication to affect the adjudication results.

14. The method of claim 1, further comprising:
perform a determination to train the at least one machine learning model based on expert feedback;
retrieve the expert feedback stored in the system; and
fine-tune the at least one machine learning model based on the retrieved expert feedback.

15. The method of claim 11, further comprising:
manipulating the plurality of data by at least one of a transformation, a filter, a modification, and a standardization;
training the adjudication and reasoning engine using the proof of achievement score; and
providing based on the proof of achievement score, one or more recommendations.

16. The method of claim 11, further comprising:
determining, based on the proof of achievement score, that a consensus is achieved; and
transmitting an achievement notification indicating the achievement of the consensus.

17. The method of claim 11, wherein the plurality of data from the one or more data sources include smart contract information concerning agreements between one or more parties.

18. The method of claim 17, wherein the smart contract information includes one or more of a healthcare token, a contractual block identifier, or a validity requirement parameter.

19. The method of claim 11, further comprising providing incentive alignment using the proof of acknowledgement result, including creating a link between a determined performance metric and one or more rewards and penalties.

20. The method of claim 11, further comprising:
monitoring data and transactions related to the transaction repository and at least one smart contract;
determining fraudulent activity is detected and issue a fraud warning to a controlling party in response; and
providing one or more reports on the data and transactions, wherein the one or more reports include at least one of patterns, trends, and improvements data.
